(19) United States
(12) Patent Application Publication
(10) Pub. No.: US 2014/0004599 A1
(43) Pub. Date: Jan. 2, 2014

(54) Anaerobic Digester Assembly and Associated Methods for Using the Same

(71) Applicant: Carey J. Boote, Holland, MI (US)

(72) Inventor: Carey J. Boote, Holland, MI (US)

(21) Appl. No.: 13/932,570

(22) Filed: Jul. 1, 2013

Related U.S. Application Data
(60) Provisional application No. 61/665,993, filed on Jun. 29, 2012.

(51) Int. Cl. C12M 1/107

(52) U.S. Cl. CPC C12M 21/04
USPC 435/290.1; 435/300.1

(57) ABSTRACT

An inexpensive, portable and modular anaerobic digester assembly, comprising: an outer container; a primary anaerobic digester container or vessel positioned within the outer container; and one or more secondary gas collection containers or vessels positioned within the outer container.

### CROSS-REFERENCE TO RELATED APPLICATIONS

This application claims the benefit of U.S. Provisional Application Ser. No. 61/665,993, filed Jun. 29, 2012, entitled “Anaerobic Digester Assembly and Associated Methods for Using the Same,” which is hereby incorporated herein by reference in its entirety—including all references and appendices cited therein.

### BACKGROUND OF THE INVENTION

### 1. Field of the Invention

The present invention relates in general to anaerobic digesters and, more particularly, to cost effective anaerobic digester assemblies which beneficially enhance portability, mobility, scalability, and modularity, among other things. The portable anaerobic digesters of the present invention are environmentally beneficial because they convert waste to resources, reduce air and water pollution, and directly contribute to the procurement of sustainable biological systems.

Anaerobic digesters have been known in the art for years and are the subject of a plurality of patents including but not limited to: U.S. Pat. No. 4,551,243 entitled “Anaerobic Digester,” U.S. Pat. No. 6,254,775 entitled “Anaerobic Digester System and Method,” U.S. Pat. No. 6,673,243 entitled “Plug Flow Anaerobic Digester,” U.S. Pat. No. 6,855,253 entitled “Anaerobic Digester,” U.S. Pat. No. 6,860,997 entitled “Apparatus and Method for Processing Organic Materials,” U.S. Pat. No. 6,921,485 entitled “Two Phase Anaerobic Organic Matter Treatment and System,” and U.S. Pat. No. 8,053,228 entitled “Bioreactor Comprising a Retaining System”—all of which are hereby incorporated herein by reference in their entirety, including all references cited therein.

U.S. Pat. No. 4,551,243 appears to disclose an anaerobic digester configuration which reduces the accumulation of undesirable solid material within the anaerobic digester. In particular, a new slurry for digesting is injected into the digester at locations extending across the horizontal width at one end, parallel to a zone of floating solids in a concentration of solid material between ten and twelve percent, with a momentum of at least 0.1 pound-foot per second. Liquid is taken out of the digester across a trough extending across the width of the digester. Bubbles are broken above the liquid level of the digester by a sieve, surfactant or combination of the two.

U.S. Pat. No. 6,254,775 appears to disclose an anaerobic digester system having a vertically upright vessel, a matrix arranged in the vessel supporting a microorganism biomass thereon, an input for supplying an input slurry of liquid and suspended solids at an upper portion of the vessel above the matrix, a gas output at the top of the vessel for withdrawing gas generated by anaerobic digestion of solids, and an effluent output at the bottom of the vessel for withdrawing liquid and remaining solids. The vessel has a preferred liquid height to diameter ratio of 2 to 1, and is constructed of inert fiberglass-reinforced plastic coated with a translucent blue gel pigment layer for filtering light at wavelengths that promote biomass cultivation. The matrix is formed as an array of panels mounted to a spindle with wheels fixed at spaced intervals along its vertical height, and the panels are made of a polyethylene grass matting providing a high surface area to volume ratio of at least 20 to 1. Gas from the top of the vessel is recycled to the bottom to generate bubbles for mixing the feedstock. The related method of anaerobic digestion includes comminuting input wastes with a slurry grinder into a pumpable slurry 8-10% by weight solids, and providing as the biomass hydrolytic bacteria, and fermentative bacteria including acetogenic and methanogenic bacteria to produce a methane gas product. Other products include an organic soil additive, bacterial solids plant food, and a filtrate used as plant tonic.

U.S. Pat. No. 6,673,243 purports to disclose a simple, reliable, inexpensive, and efficient anaerobic digester for treating organic wastes at a shortened residence time. The anaerobic digester is a multi-chambered digester that can handle wastewater and sludge in a large volume at a high flow rate. The digester also allows collection of methane gas for use as an energy source. The reactor is based on a sequential series of reaction chambers in a design that does not require internal moving parts. The volume of the chambers is adjusted to control the relative residence time of the waste to select an anaerobic microorganism group or groups that can efficiently digest the waste presented to that chamber. Under most conditions, no addition of bacteria is necessary. The digester works efficiently using microbes native to the waste material. After the reaction chambers and just prior to leaving as effluent, a settling chamber is located to reclaim the microbes and remove additional solids. In one embodiment, the reactor comprises four sequential chambers. However, other chamber numbers and geometries will achieve the same result if the residence time in each chamber is properly adjusted. Neither pH nor temperature needed to be controlled. However, for a higher yield of methane, pH may be controlled from about 6 to about 8.

U.S. Pat. No. 6,855,253 appears to disclose an anaerobic digester and a method for treating organic waste and recovering a usable quality methane gas. A first cover of gas permeable material conducive to bacterial colonization covers the surface of a slurry of organic waste material in a containment vessel. The permeable cover acts as a media to support and encourage the growth of methanogenic bacteria. A gas collection apparatus is installed on the first cover. A second cover of gas impermeable material is installed over the gas collection apparatus. A gas collection space is formed between the two covers. Edges of the first and second covers are closed to inhibit the escape of gas from the gas collection space. Biogas produced as a result of anaerobic digestion activity permeates the gas permeable cover and enters the collection space. From the collection space the gas is drawn into the gas collection apparatus which can, for example, be comprised of a network of perforated ducts connected to a blower that draws gas out of the ducts for delivery to a storage or usage location.

U.S. Pat. No. 6,860,997 appears to disclose a method and apparatus for processing organic materials by anaerobic digestion. The apparatus comprises a first covered fluid container, such as a retention pond covered with an oxygen impervious membrane. An aqueous slurry comprising anaerobically digestible fiber is disposed within the first container. A second covered container is disposed within or adjacent to the first container. The second covered container comprises a second upper section and a second lower section. A fluid inlet in the second upper section receives fluid flow from the first container, where a screen is disposed between the first container and the second container, across the fluid inlet. The fluid flow across the screen causes the formation of a filter cake on the screen as fluid flows across the screen, allowing for filtration of the aqueous slurry, and separation of liquids and gas as those components enter the second container. Each apparatus unit may be considered a module. A digester system may be formed by connecting a plurality of the modules together in parallel or in series.

U.S. Pat. No. 6,921,485 appears to disclose a process and system having an organic matter influent introduced into an acid reactor. The acid reactor is maintained under conditions to facilitate creation of volatile acids in a fluid having water and suspended solids forming a sludge effluent. The sludge effluent is communicated to an acid separation element where the water and volatile acids are separated from the suspended solids. A liquid stream may be communicated from the acid separation element to a methane reactor and a solids recycle stream are communicated to the acid reactor. The methane reactor may be maintained under conditions to facilitate creation of biogas. A liquid effluent is communicated from the methane reactor to a methane separation element where liquid water is separated from the solids. A second solids recycle is communicated from the methane separation element to the methane reactor.

U.S. Pat. No. 8,053,228 appears to disclose a bioreactor in which a supporting wall or a retaining mechanism that represents pressure relief for a gas-tightly embodied flap is provided behind the flap used for filling and emptying the bioreactor while percolate is prevented from accumulating between the gas-tight flap and the retaining mechanism. The gas-tightly closable flap, which has a sufficiently large size, makes it easy to fill biomass into the container and remove the remaining biomass following the methanation process. The biomass applies substantial or at least a certain amount of pressure to the retaining system when the container is closed, whereby the flap is relieved of the load and can be embodied in a light and accurately sealing manner. A device for draining percolating juice or percolate is disposed in the floor and/or the walls of the bioreactor between the flap and the retaining system such that percolating juice is prevented from accumulating in the zone between the flap and the retaining system or percolate located there can be pumped off before the flap is opened.

While various configurations of anaerobic digesters have been known in the art for years, issues associated with cost, portability, mobility, scalability, and modularity, among other things, remain largely problematic.

Therefore, it is an object of the present invention to provide anaerobic digester assembly configurations and associated methods which remedy the aforementioned drawbacks associated with current anaerobic digester systems.

These and other objects of the present invention will become apparent in light of the present specification, claims, and drawings.

### SUMMARY OF THE INVENTION

In one embodiment, the present invention is directed to an inexpensive, portable and modular anaerobic digester assembly comprising: (1) an outer container; (2) a primary anaerobic digester container or vessel positioned within the outer container; and (3) one or more secondary gas collection containers or vessels positioned within the outer container.

In a preferred embodiment of the present invention, the at least one secondary gas collection vessel comprises two secondary gas collection vessels.

In another preferred embodiment of the present invention, the anaerobic digester assembly further comprises an isolator positioned both within the outer container and between the primary anaerobic digester vessel and the at least one secondary gas collection vessel.

In yet another preferred embodiment of the present invention, the anaerobic digester assembly further comprises containment regulating insulation positioned with the outer container.

In another aspect of the present invention, the anaerobic digester further comprises a water heater and/or a methane generator associated with the at least one secondary gas collection vessel.

In one embodiment, the present invention is also directed to an anaerobic digester assembly comprising: (1) an outer container; (2) a primary anaerobic digester vessel positioned within the outer container; (3) at least one secondary gas collection vessel positioned within the outer container; (4) an input vessel, wherein the input vessel comprises at least one of pre-anaerobically digested animal excrement and partially anaerobically digested animal excrement; (5) an output vessel, wherein the output vessel comprises anaerobically digested animal excrement; (6) an input valve, wherein the input valve is in fluid communication with the input vessel and the primary anaerobic digester vessel; and (7) an output valve, wherein the output valve is in fluid communication with the output vessel and the primary anaerobic digester vessel.

In another embodiment, the present invention is further directed to an anaerobic digester assembly comprising: (1) an outer container; (2) a primary anaerobic digester vessel positioned within the outer container; (3) at least one secondary gas collection vessel positioned within the outer container; (4) an input vessel, wherein the input vessel comprises at least one of pre-anaerobically digested animal excrement and partially anaerobically digested animal excrement; (5) an output vessel, wherein the output vessel comprises anaerobically digested animal excrement; (6) an input valve, wherein the input valve is in fluid communication with the input vessel and the primary anaerobic digester vessel; (7) an output valve, wherein the output valve is in fluid communication with the output vessel and the primary anaerobic digester vessel; (8) an isolator, wherein the isolator is positioned both within the outer container and between the primary anaerobic digester vessel and the at least one secondary gas collection vessel; (9) containment regulating insulation positioned with the outer container; (10) a water heater, wherein the water heater is associated with the at least one secondary gas collection vessel; and (11) a methane generator, wherein the methane generator is associated with the at least one secondary gas collection vessel.

### BRIEF DESCRIPTION OF THE DRAWINGS

Certain embodiments of the present invention are illustrated by the accompanying figures. It will be understood that the figures are not necessarily to scale and that details not necessary for an understanding of the invention or that render other details difficult to perceive may be omitted. It will be further understood that the invention is not necessarily limited to the particular embodiments illustrated herein.

The invention will now be described with reference to the drawings wherein:

FIG. 1 of the drawings is a schematic representation of an anaerobic digester assembly manufactured in accordance with the present invention.

### DETAILED DESCRIPTION OF THE INVENTION

While this invention is susceptible of embodiment in many different forms, there is shown in the drawings and described herein in detail several specific embodiments with the understanding that the present disclosure is to be considered as an exemplification of the principles of the invention and is not intended to limit the invention to the embodiments illustrated.

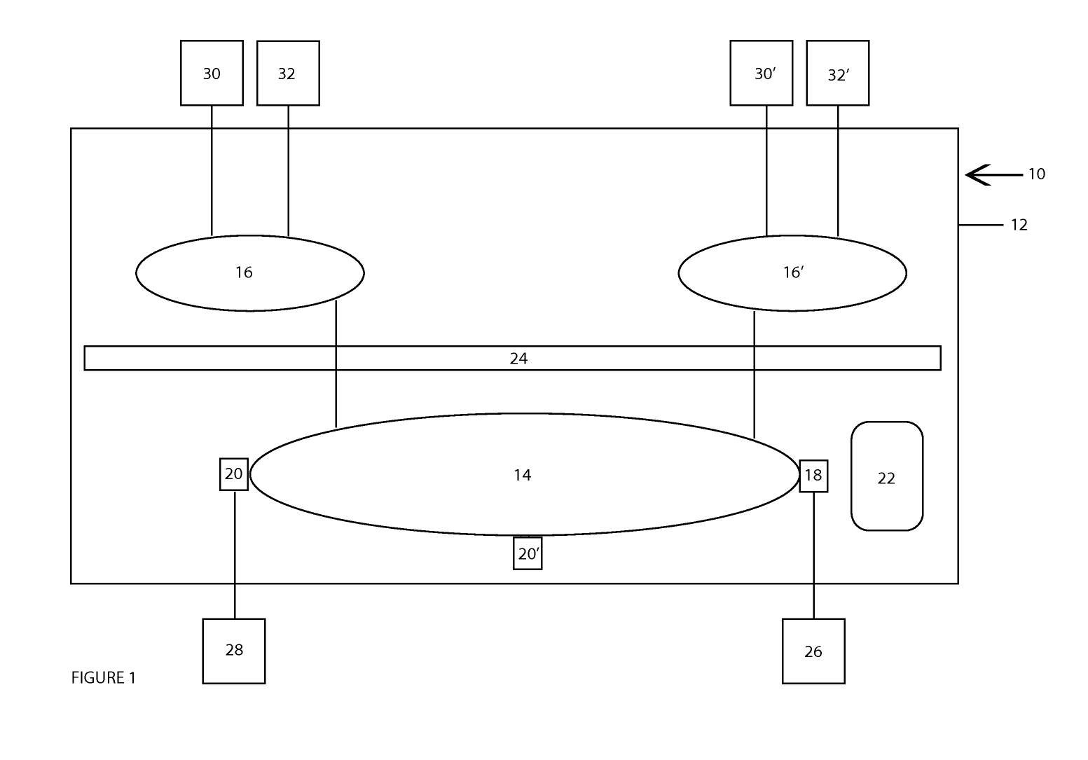

It will be understood that like or analogous elements and/or components, referred to herein, may be identified throughout the drawings with like reference characters. It will be further understood that FIG. 1 is merely a schematic representation of an anaerobic digester assembly. As such, some of the components have been distorted from their actual scale for pictorial clarity.

In accordance with the present invention, anaerobic digester assemblies disclosed herein are configured for use in a plurality of applications including, but not limited to, small, medium, and large horse, dairy and/or other animal farms. The anaerobic digester assemblies of the present invention, among other benefits, are inexpensive compared to traditional anaerobic digesters. The anaerobic digester assemblies of the present invention are also substantially more portable, mobile, scalable, and/or modular, relative to current commercially available anaerobic digesters. Moreover, the anaerobic digester assemblies of the present invention facilitate the conversion of waste material to a resource which is biodegradable, compostable, and sustainable with respect to biological systems.

Referring now to the drawings and to FIG. 1 in particular, a schematic representation of anaerobic digester assembly 10 is shown which generally comprises outer container 12, primary anaerobic digester container or vessel 14, and one or more secondary gas collection containers or vessels 16.

Outer container 12 preferably comprises, for example, a 20 or 40 foot shipping container that is commercially available from Aztec Container in the United States. Outer container 12 preferably includes one or more apertures or doors for receiving primary anaerobic digester container or vessel 14, and one or more secondary gas collection containers or vessels 16.

Primary anaerobic digester container 14 preferably comprises a flexible, multi-layer plastic enclosure or bag which is commercially available from Holland Storage Systems, LLC in Holland, Mich. It will be understood that while primary anaerobic digester container 14 has been disclosed for illustrative purposes only as comprising a flexible, multi-layer plastic enclosure, non-flexible, semi-flexible, and/or flexible enclosures are likewise suitable for use in accordance with the present invention. Primary anaerobic digester container 14, either alone, or in combination with a heating system, (e.g., a hot water coil and/or bag, etcetera) preferably controllably maintains any contents therein at approximately 100 degrees Fahrenheit for maximizing methane production. Primary anaerobic digester container 14 also preferably includes one or more input valves 18 and one or more output valves 20.

One or more secondary gas collection containers or vessels 16 preferably comprise a flexible, multi-layer plastic enclosure or bag which is commercially available from Holland Storage Systems, LLC in Holland, Mich. It will be understood that while one or more secondary gas collection containers or vessels 16 preferably has been disclosed for illustrative purposes only as comprising a flexible, multi-layer plastic enclosure, non-flexible, semi-flexible, and/or flexible enclosures are likewise suitable for use in accordance with the present invention—the only limitation being that secondary gas collection container(s) or vessel(s) 16 must be at least substantially impermeable to methane gas.

Anaerobic digester assembly 10 optionally includes temperature, smell, sound, and/or contaminant regulating insulation 22 which is preferably positioned in any void space within outer container 12.

Anaerobic digester assembly 10 also optionally includes isolator 24 which is preferably positioned between primary anaerobic digester container or vessel 14 and one or more secondary gas collection containers or vessels 16.

In one embodiment anaerobic digester assembly 10 further comprises gas, temperature, moisture, and/or flow rate sensors which are optionally in communication with a process and/or safety system.

In another preferred embodiment of the present invention, anaerobic digester assembly 10 further comprises a pressure relief valve associated with at least one of outer container 12, primary anaerobic digester container or vessel 14, and one or more secondary gas collection containers or vessels 16.

In operation, primary anaerobic digester container 14 is preferably supplied, via input valve 18, with animal excrement (e.g., cow, cattle, goat, sheep, giraffe, bison, moose, elk, yak, water buffalo, deer, camel, alpaca, llama, antelope, pronghorn, nilgai, etcetera) from input vessel 26. It will be understood that the solids content of the excrement is preferably approximately 15% by weight. After undergoing anaerobic digestion, the animal excrement is preferably transferred to output vessel 28. During the anaerobic digestion process, methane is collected in one or more secondary gas collection containers or vessels 16 which can be subsequently utilized with water heater 30 and/or produce energy via, for example, methane generator 32. The anaerobically digested solids are preferably utilized as a fibrous material in an eco-friendly product. The residual liquid is preferably utilized as nitrogen rich fertilizer. It will be understood that the anaerobically digested material may be further and/or alternatively utilized in any one of a number of other eco-friendly manners.

The foregoing description merely explains and illustrates the invention and the invention is not limited thereto except insofar as the appended claims are so limited, as those skilled in the art who have the disclosure before them will be able to make modifications without departing the scope of the invention.

What is claimed and desired to be secured by Letters Patent of the United States is:

1. An inexpensive, portable and modular anaerobic digester assembly, comprising:
an outer container;
a primary anaerobic digester vessel positioned within the outer container; and
at least one secondary gas collection vessel positioned within the outer container.

2. The anaerobic digester assembly according to claim 1, wherein the at least one secondary gas collection vessel comprises two secondary gas collection vessels.

3. The anaerobic digester assembly according to claim 1, further comprising an isolator positioned both within the outer container and between the primary anaerobic digester vessel and the at least one secondary gas collection vessel.

4. The anaerobic digester assembly according to claim 1, further comprising containment regulating insulation positioned with the outer container.

5. The anaerobic digester assembly according to claim 1, further comprising a water heater associated with the at least one secondary gas collection vessel.

6. The anaerobic digester assembly according to claim 1, further comprising a methane generator associated with the at least one secondary gas collection vessel.

7. An anaerobic digester assembly, comprising:
an outer container;
a primary anaerobic digester vessel positioned within the outer container;
at least one secondary gas collection vessel positioned within the outer container;
an input vessel, wherein the input vessel comprises at least one of pre-anaerobically digested animal excrement and partially anaerobically digested animal excrement;
an output vessel, wherein the output vessel comprises anaerobically digested animal excrement;
an input valve, wherein the input valve is in fluid communication with the input vessel and the primary anaerobic digester vessel; and
an output valve, wherein the output valve is in fluid communication with the output vessel and the primary anaerobic digester vessel.

8. An anaerobic digester assembly, comprising:
an outer container;
a primary anaerobic digester vessel positioned within the outer container;
at least one secondary gas collection vessel positioned within the outer container;
an input vessel, wherein the input vessel comprises at least one of pre-anaerobically digested animal excrement and partially anaerobically digested animal excrement;
an output vessel, wherein the output vessel comprises anaerobically digested animal excrement;
an input valve, wherein the input valve is in fluid communication with the input vessel and the primary anaerobic digester vessel;
an output valve, wherein the output valve is in fluid communication with the output vessel and the primary anaerobic digester vessel;
an isolator, wherein the isolator is positioned both within the outer container and between the primary anaerobic digester vessel and the at least one secondary gas collection vessel;
containment regulating insulation positioned with the outer container;
a water heater, wherein the water heater is associated with the at least one secondary gas collection vessel; and
a methane generator, wherein the methane generator is associated with the at least one secondary gas collection vessel.
